(19) United States
(12) Patent Application Publication
(10) Pub. No.: US 2005/0084546 A1
(43) Pub. Date: Apr. 21, 2005

(54) Antioxidant and antiactinic tea plant products

(76) Inventors: Walter P. Smith, Woodbury, CT (US); Joanne Ng, Port Washington, NY (US)

Correspondence Address: ANTHONY H. HANDAL;KIRKPATRICK & LOCKHART NICHOLSON GRAHAM LLP, 599 LEXINGTON AVENUE, 33RD FLOOR, NEW YORK, NY, 10022-6030, US

(21) Appl. No.: 10/687,565

(22) Filed: Oct. 16, 2003

(51) Int. Cl. A61K 35/78; A23F 3/00

(52) U.S. Cl. 424/729; 426/597

(57) ABSTRACT

Disclosed is a composition of white, green and yellow teas, in a preferred weight proportion of about 5:3:2 which has surprising ability to prevent damage caused by ultraviolet radiation as well as being a strikingly effective antioxidant. The mixed tea blend is useful in topical cosmetics or dermatologics, in beverage preparation and as a foodstuffs ingredient.

The present invention relates to novel tea plant products having antioxidant and antiactinic activities. More particularly, the invention relates to novel tea extracts and dry tea blends and to beverages, foodstuffs and topical cosmetic and dermatological compositions containing or prepared from the inventive tea extracts and dry tea blends.

Tea products have long been considered beneficial to human health and well-being. An extensive international industry flourishes on products derived from the tea plant, Camellia sinensis, which provides the hot and cold beverages that are a daily staple for hundreds of millions of people around the world. Tea is widely enjoyed considered relaxing, and is often the focus of pleasant social rituals and is scientifically known to have antioxidant and stimulant properties.

Herbal teas have for centuries been made from many plants other than Camellia sinensis, chamomile and peppermint being just two examples. To avoid confusion with teas from the tea plant herbal teas are often called “infusions”. References to “tea” herein are to be construed as references to the tea plant, Camellia sinensis, and its products or to equivalent plants, for example Camellia assaimica, or their hybrids, and the products of such equivalent plants, rather than to unrelated herbal tea products such as chamomile and peppermint.

The overwhelming majority of commercial tea products comprise black teas prepared by aeration, and possibly abrasion, of the leaves of the green tea plant conducted for time sufficient time to change the leaves' color from green to copper and to intensify their flavor. This is a fermentation process wherein various natural chemical reactions, including enzymatic oxidation, occur. Black teas are sold as the dry packaged tea product, as bottled and canned iced teas, and are commercially dispensed as hot and cold beverages in tremendous volumes every day.

Green tea products are another important commercial category. Green teas are produced from the plant without extended air exposure, by merely allowing the plant to wither and dry. More popular in Asia than the West, green teas and green tea products are similarly staples of commerce and are additionally used as flavorings in foodstuffs, for example in green tea ice cream.

In recent years, green tea has become recognized for having valuable antioxidant properties, which have been associated with organic constituents such as polyphenols and catechins., leading to the development of standardized aqueous extracts and the employment of green tea extracts in cosmetic and dermatologic products. Such recognition of the neutraceutical or therapeutic benefits of green tea has been accompanied by a more widespread appreciation of the potential benefits to be obtained from biologically active botanicals.

Chang in U.S. Pat. No. 5,043,100 discloses production of tea-derived oil_soluble antioxidants by the vacuum steam distillation of alcohol extracts of spent black tea or spent green tea. The antioxidant constituents and properties of black and green teas are discussed and a substantial bibliography of the art at that time is provided.

Ekanayake in U.S. Pat. No. 5,879,733 discloses green tea extracts described as having improved clarity and color which are obtained by metal cation removal and nanofiltration of the extract. As disclosed by Ekanayake, the extraction of tea material is well known in the art. By way of example, green tea can be extracted with hot or cold water to form a dilute extract containing soluble tea solids which can be concentrated to and sold in frozen, refrigerated or dried form. Such methods are also described in Ekanayake.

Barmentlo, et al. in U.S. Pat. No. 5,258,188 disclose some other processes of preparing tea extracts.

McCook in U.S. Pat. No. 5,306,486 discloses the use of green tea in cosmetic sunscreen compositions. Green tea constituents, the fermentation process and the preparation of extracts are described as are various cosmetic formulations including green tea extracts.

Xiong, et al. in U.S. Pat. No. 6,299,925 discloses an effervescent green tea extract formulation.

It would be desirable to provide new tea products having beneficial properties that are suitable for commercial exploitation.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context.

### BRIEF SUMMARY OF THE INVENTION

The present invention provides a mixed tea composition comprising:
a) from about 30 to about 70 weight percent of a white tea;
b) from about 15 to about 50 weight percent of a green tea;
c) from about 5 to about 40 weight percent of a yellow tea; the percentage weights being percentages of the total weight of the mixed teas in the composition.

Preferably, each tea comprises an aqueous extract of the respective tea. Each tea extract can have a predetermined potency as indicated by the concentration of a marker compound. The marker compound can be caffeine, or other suitable active ingredient, for example theobromine, optionally at a concentration of about 1 percent by weight of the extract.

In one embodiment the proportion of each tea is at least about 20 weight percent based upon the total weight of the mixed tea composition.

Another preferred composition comprises:
a) from about 40 to about 60 weight percent of the white tea;
b) from about 25 to about 35 weight percent of the green tea;
c) from about 15 to about 25 weight percent of a yellow tea; the percentages being percentages of the total weight of the mixed tea extracts in the composition.

The invention also provides a mixed tea composition comprising proportions of white tea, green tea and yellow tea effective to provide inhibition of ultaviolet-induced cell renewal approximately equivalent to that provided by an SPF sunscreen or effective to provide any of the antioxidant or antiactinic properties described quantitatively herein.

In addition, the invention provides a human-consumable product having from about 0.01 to about 10 weight percent of a mixed tea composition according to claim 1 which product can be selected from the group consisting of topical cosmetic compositions, topical dermatological compositions, sunscreens, beverages and foodstuffs.

### DETAILED DESCRIPTION OF THE INVENTION

In contrast to the art which has focused attention on the preparation and properties of black tea extracts primarily for the production of beverages and has looked to the therapeutic and prophylactic properties of green tea extracts, the present invention considers some alternative tea materials and discovers new compositions including such alternative tea materials which have unexpected beneficial properties that are useful, inter alia, in topical cosmetic compositions.

Yellow teas are prepared by partial fermentation of the raw green tea product, substantially less than would produce a black tea. White teas are prepared from new buds harvested before they open which are withered and gently dried.

Surprisingly, it has been discovered pursuant to the present invention, that novel compositions having both antioxidant and antiactinic properties can be provided by combining aqueous extracts of green, yellow and white teas. The aqueous extracts may be made by any suitable method, as known to those skilled in the art, employing as solvent water, optionally adjusted to a desired pH, or a water-alcohol mixture or other suitable aqueous mixture. Preferred embodiments of such novel compositions exhibit surprising abilities to prevent biological damage caused by ultraviolet and solar radiation.

### EXAMPLE 1

### Tea Extracts

Aqueous extracts of white, green and yellow teas are prepared by steeping dried tea leaves in boiling water, filtration and adjustment to about 1% caffeine content by weight, as determined by high pressure liquid chromatography, or other known method. The caffeine content serves as a marker indicative of potency. Satisfactory results are obtainable by adjusting the caffeine content to be in the range of from about 0.8 to about 1.2 percent by weight.

### Example 2

### Mixed Tea Extract, Leaf Mixing

An aqueous mixed tea extract of white, green and yellow teas is prepared by steeping a mixture of the dried leaves of the three teas in a respective weight proportion of 5:3:2 in boiling water, filtration and adjustment to a desired potency, as described in Example 1.

### Example 3

### Mixed Tea Extract, Extract Mixing

An aqueous mixed tea extract of white, green and yellow tea is prepared by mixing the three extracts prepared by the method of Example 1 in a proportion of 50 parts by weight white tea, 30 parts by weight green tea and 20 parts by weight yellow tea. The resulting mixed tea blend is diluted with water to a concentration of about 2-3 percent by weight of extract and employed in the following tests, as noted.

### Test 1. In Vitro Antioxidant Activity: (a) Cytochrome C Oxidation

An in vitro cytochrome C oxidation-reduction assay conducted in the presence of isolated neutrophils is used to assess the antioxidant activity of a group of test materials comprising aqueous extracts of the inventive mixed tea blend prepared by the method of Example 3 having the concentrations indicated in Table 1, below, and known antioxidant materials, namely green tea extract, white tea extract, grape polyphenols and vitamin E, as controls. Reduction of cytochrome C by free radicals causes an increase in light absorbence at 550 nm. Cytochrome C is an ubiquitous iron-containing cellular respiratory enzyme. Singlet oxygen prevents free radical reduction and the consequent increase in light absorbance. The oxidation-reduction assay determines the ability of the test materials to control the activity of chemically generated singlet oxygen, permitting the reductive free radicals to induce increased light absorbence, which is measured. The antioxidant assay results are set forth in Table 1, below.

TABLE 1
Inhibition of Cytochrome C Oxidation

| Test Material | 0.1 μg/ml | 1.0 μg/ml | 10 μg/ml | 25 μg/ml |
|---|---|---|---|---|
| Mixed Tea Blend* | 64% | 78% | 95% | 94% |
| Green Tea | 23% | 45% | 67% | 71% |
| WhiteTea | 17% | 38% | 33% | 25% |
| Grape Polyphenols | 0% | 11% | 24% | 33% |
| Vitamin E | 21% | 15% | 11% | 14% |

As indicated in line 1, at a concentration of 10 μg/ml the mixed tea blend shows a 95% inhibition of cytochrome C oxidation, a comparable inhibitory activity at the higher concentrations of 25 μg/ml and a strong inhibition of 78% at the lower concentration of 1.0 μg/ml. As may be seen by comparing the data in line 1 with the data in lines 2-5, the mixed tea blend is surprisingly more effective than green tea or white tea extracts used alone and is more effective than grape polyphenols or vitamin E at all test concentrations, in inhibiting singlet oxygen induced oxidation.

The assay system employed, determination of the impact of singlet oxygen on a biological entity, cytochrome C, is believed to be a useful model of in vivo activity. However, the invention is not dependent upon this or any other theory.

### Test 2. Free Radical Cytotoxicity Inhibition

Xanthine oxidase and hypoxanthine, referenced “XO” in Table 2, are employed together as a system to generate free radicals such as hydrogen peroxide, the hydroxyl radical and superoxide, as is known in the art. The free radical products of xanthine oxidase and hypoxanthine referenced “ROS” hereinafter, provide a test system which can be used to model the ability of test materials to protect biological organisms against the action of free radicals.

Test 2 determines the ability of the group of test materials employed in Test 1 to reduce ROS cytotoxicity to fibroblasts and keratinocytes. Cells are seeded in 96-well plates and grown for two days. They are then exposed for about 24 hours to hypoxanthine at 100 □ g/ml and to various concentrations of xanthine oxidase, ranging, as shown in Table 2's column headers, from 2 mU/ml to 40 mU/ml. Fresh culture medium is added after exposure to ROS and cell death is assessed by the conventional MTT dye method wherein light absorbence at 570 nm is measured. Only living cells take up the MTT dye which absorbs at the measurement wavelength. The cytotoxicity test results, using the quantity of the test materials listed to inhibit free radical toxicity, are set forth in Table 2, below. The control is an aliquot of water.

TABLE 2
Cell Survival after Exposure to ROS Free Radicals

| Test Material | XO 2 mU | XO 10 mU | XO 20 mU | XO 40 MU |
|---|---|---|---|---|
| Control (water) | 56% | 23% | 0% | 0% |
| Mixed Tea Blend | 98% | 89% | 66% | 35% |
| Grape | 92% | 67% | 34% | 22% |
| polyphenols | | | | |
| Vitamin E | 89% | 47% | 25% | 13% |
| Green Tea | 95% | 72% | 41% | 31% |

Referring to line 2 of Table 3, it can be seen that the inventive mixed tea blend effectively inhibits the damaging effects of the ROS generated by the xanthine system, at test concentrations up to 20 mU of the xanthine oxidase system. Even at the relatively intense concentration of 40 mU there is some inhibition. The mixed tea blend is more effective than each of the other materials tested at all concentrations. While both green tea and grape extract, materials rich in polyphenols, show good activity, the mixed tea blend is significantly more protective than either tea on its own at the important intermediate concentrations of 10 and 20 mU.

### Test 3. UV Cytotoxicity Assay

Fibroblasts, grown in Dulbecco medium (DM) supplemented with fetal calf serum are plated in 24 well plates at a density of 20,000 cells per well. Twelve hours after plating, fresh DM with fetal calf serum is added. After an additional 12 hours various test materials as set forth in Table 3, are added to the wells. Six, twelve and twenty four hours later, cultures are exposed to UV at varying low and high doses. The low dose is chosen to stimulate growth and cause a hyper-proliferative response, while the higher dose is chosen to induce cytotoxic effects. After 72 and 144 hours, cells are trypsinized, and counted in a hemocytometer chamber. The cytotoxicity test results using the indicated quantities of the test materials to inhibit free radical toxicity, are set forth in Table 3, below. The control is an aliquot of water.

TABLE 3
Cell Survival after Exposure to UV

| Test Material | 1 mJ/cm2 | 2 mJ/cm2 | 5 mJ/cm2 | 10 mJ/cm2 |
|---|---|---|---|---|
| Control (water) | 67% | 44% | 32% | 12% |
| Mixed Tea Blend | 100% | 78% | 75% | 67% |
| Grape polyphenols | 88% | 65% | 63% | 50% |
| Vitamin E | 65% | 42% | 35% | 20% |
| Green Tea | 76% | 55% | 50% | 33% |

The inventive mixed tea blend was significantly more effective than all the other test materials at all UV dosages, with the protective effects, in most cases, becoming more pronounced at higher UV dosages. At the highest dosage, 10 mJ/cm², the mixed tea blend provided very substantial 67% cell survival, whereas the best comparative test material, grape polyphenols provided only 50%.

### Test 4. In Vitro Antioxidant Activity: (c) Reduction of Lipid Peroxidation

The ability of test materials to reduce lipid peroxidation in vivo is assessed by applying various topical formulations to each forearm of human test subjects for a period of two weeks. Five times per week subjects sit in noon time sun for at least one hour with their volar forearms exposed and facing toward the incident sunlight. Lipids are extracted from the skin surface over a defined 10 cm² area by successive washing with non-polar (hexane) and polar solvents (ethanol). The extracts are pooled and lipid peroxide values are obtained via standard methods. These results are shown in Table 4, below.

TABLE 4
Reduction of Lipid Peroxidation (Units are peroxide values)

| | Before | After 1st | After |
|---|---|---|---|
| Test Cell | treatment | application* | 2 weeks** |
| Control - No Treatment | 4.16 | 4.72 | 5.68 |
| 2% Mixed Tea Blend | 4.23 | 4.54 | 5.03 |
| (immediately before exposure) | | | |
| Control - SPF 15 sunscreen | 4.31 | 4.37 | 4.84 |

*After 1 UV exposure,
**after 10 UV exposures

Referring to Table 4, the lipid peroxide data after the first application indicates lipid peroxidation or damage, is increased by about 15% (4.16 to 4.72) after 1 UV exposure in the no treatment control and rather less for the skin protected by the inventive mixed tea blend or the SPF 15 sunscreen control. However, after 10 exposures over 2 weeks, lipid damage increases by about 30% (4.16 to 5.68). The SPF 15 sunscreen shows a damage reduction of about 66% (4.31 to 4.84 a change of only 0.53 units as compared to 1.52 units for the no treatment control). The 2% tea also effects substantial damage reduction of about 47% (4.23 to 5.03, a change of only 0.80 units as compared to 1.52 units for the no treatment control). Thus, the mixed tea blend exhibits significant ability to inhibit UV-induced lipid peroxidation damage, albeit not quite as much as an SPF 15 sunscreen. Protection against peroxidation has valuable clinical potential because oxidized lipids result in altered barrier properties, increased trans-epidermal water loss (“TEWL”), dryness and irritated skin.

### Test 5. In vivo Inhibition of UV-Induced Skin Cell Renewal

The dansyl chloride staining technique is used to measure rates of skin cell turnover, under normal conditions and under conditions of exposure to ultraviolet light with and without protection from various test materials.

Summarizing known procedural methods, the stratum corneum is stained with fluorescent dansyl chloride by applying semi-occlusive patches of 5% dansyl chloride milled into petrolatum for 24 hours. After assuring that the stain is completely taken up by the stratum corneum layers by viewing under a quartz mineral lamp, subjects are instructed to apply water or the test product. Skin sites are treated with the various doses of UV indicated in MED in Table 6A, below, 3 days prior to and 1 day after patching with dansyl chloride. Visual inspection of the sites under UV lamp is made until the stain disappears reflecting the time required for complete turnover of the full thickness stratum corneum. Changes in cell renewal due to test material effects and UV exposure can be expressed as a percentage change compared to water-treated or untreated controls. The results are shown in Table 5, below.

TABLE 5
Effect of UV Exposure on Cell Renewal Rates

| | | % Change (Decrease in |
|---|---|---|
| Test Cell | Turnover (Days) | turnover times) |
| No exposure | 17.5 | NA |
| 0.5 MED-2 doses | 17.2 | 1.7% |
| 1.0 MED-2 doses | 14.7 | 16% |
| 2.0 MED-2 doses | 10.2 | 41.7% |

As shown in line 2, UV exposure of less than 0.5 MED has little effect on observed cell turnover rates. However exposures of 1 and 2 MED significantly reduce the number of days required for stain removal indicating a hyperproliferative effect amounting to increases in renewal by more than 40%. These data provide a comparative basis for assessment of the UV-protective effects of the test materials results of which are shown in Table 5A, below.

TABLE 5A
In vivo Inhibition of UV-Induced Skin Cell Renewal

| | No UV | | Hyperproliferative |
|---|---|---|---|
| Test Cell | Exposure | UV Exposure | Effect** |
| Control | 17.6 | 10.2 | 41.7% |
| SPF 15 | 17.4 | 14.8 | 14.9% |
| 5% Vitamin E | 18.1 | 10.5 | 41.9% |
| 5% Green Tea | 15.6 | 11.0 | 29.5% |
| 3% Mixed Tea Blend | 15.9 | 13.5 | 15.1% |
| SFF 15 & 3% Mixed | 15.4 | 15.5 | 0% |
| Tea Blend | | | |

The results in Table 5A demonstrate the ability of the various test materials to counteract UV-induced hyperproliferation. Treatment with a conventional SPF 15 sunscreen (Banana Boat; titanium dioxide with chemical filters), while eliminating any visible effects of the UV provided only limited protection against hyperproliferation, increases of about 15% being observed. Green tea and vitamin E are also tested, lines 3-4 with poor results. Vitamin E at 5% in an oil-based formulation appears to provide no protection against UV-induced hyperproliferation while green tea, at 5% concentration, has only a modest effect which is substantially less than that of the SPF sunscreen, reducing hyperproliferation rates only to about 30%. In surprising contrast, the inventive mixed tea blend at 3% concentration, used alone, protects against hyperproliferation almost as effectively as does the SPF 15 sunscreen (line 5). Remarkably, and quite unforeseeably, when the inventive mixed tea blend is combined with the SPF 15 sunscreen, substantially complete protection is observed as no hyperproliferation is seen.

### Test 6. Clinical Studies of Inhibition of UV-Induced Facial Symptoms

In a four week clinical study, twenty subjects ages 35-55 are exposed to 1MED artificial UV three times per week for four weeks to one side of the face randomized. Half the subjects applied a placebo gel (0.2% carbopol 940, 1% propylene glycol, preserved with 0.1% methyl paraben, pH adjusted to 6-7) and the other half applied a mixed tea blend product comprising the same gel containing 2% mixed tea blend product prepared as described in Example 3. The products are applied twice daily, a.m. and p.m., consistently with standard product application. After 4 weeks, and at the start of the study, the subjects are evaluated clinically and with bio-instrumentation. Clinical evaluations are made of dryness, skin scaliness, erythema and skin peeling. Instrumental analyses are made of skin redness (a*-value Minolta Meter), skin desquamation (sebu-tape), skin dryness (Minolta Meter). The evaluations and analyses are made by methods known to those skilled in the art. The results are shown in Table 6, below.

TABLE 6
Clinical Study Inhibition of UV-Induced Facial
Symptoms

| | Placebo | 2% Blend |
|---|---|---|
| Parameter | % Increase | % Increase |
| Dryness (Clinical) | +22 | +7 |
| Scaliness (Clinical) | +35 | +3 |
| Erythema (Clinical) | +67 | +11 |
| Peeling (Clinical) | +17 | −6 |
| Redness (Minolta Meter) | +44 | +17 |
| Desquamation | +104 | +11 |
| Dryness (Nova Meter) | +56 | +10 |

Referring to Table 6, the results indicate that 2 weeks of exposure to artificial UV resulted in a significant deterioration of the skin condition, in all the categories examined, on the faces treated with placebo. On the faces treated with the gel containing the inventive mixed tea blend the negative are largely prevented. Dryness is reduced to a low level whether measured clinically or optically. Skin, scaliness, peeling and desquamation are virtually eliminated. Clinical erythema and optically measured redness are also reduced to low levels. Thus, the inventive mixed tea blend provides remarkably effective inhibition of UV-induced skin damage, as indicated in these clinical tests.

Compositions of the present invention may take many forms, as will be understood by those skilled in the art. Some suitable compositions are set forth in McCook U.S. Pat. No. 5,306,486 including either solid or liquid, aqueous or anhydrous and opaque or transparent compositions especially cosmetic compositions in emulsion form. An emulsion is a dispersed system containing at least two immiscible liquid phases, one of which is dispersed in the form of small droplets throughout the other. Water and oil are the most common immiscible phases. An emulsion in which oil is dispersed as droplets throughout the aqueous phase is termed an oil_in_water emulsion. When water is the dispersed phase and an oil is the dispersion medium, a water_in_oil emulsion exists. Contemplated within the scope of this invention are emulsions in the form of lotions and creams of both types of emulsions, those where the water phase is continuous and those where the oil phase is continuous. The amount of these phases may range from about 99:1 to 1:99 by weight.

The term pharmaceutically acceptable carrier is intended to include emollients, surfactants, humectants and water. Total amount of the carrier may range from about 30 to about 99.9%, preferably from about 50 to about 90%, optimally from about 70 to about 85% by weight.

A variety of oily emollients may be employed in the compositions of this invention. These emollients may be selected from one or more of the following classes:
1. Hydrocarbon oils and waxes. Examples thereof are mineral oil, petrolatum, paraffin, ceresin, ozokerite, microcrystalline wax, polyethylene, and perhydrosqualene.
2. Triglyceride esters such as vegetable and animal fats and oils. Examples include castor oil, cocoa butter, safflower oil, cottonseed oil, corn oil, olive oil, cod liver oil, almond oil, avocado oil, palm oil, sesame oil, squalane, and soybean oil.
3. Acetoglyceride esters, such as acetylated monoglycerides.
4. Ethoxylated glycerides, such as ethoxylated glyceryl monostearate.
5. Alkyl esters of fatty acids having 10 to 20 carbon atoms. Methyl, isopropyl, and butyl esters of fatty acids are useful herein. Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate.
6. Alkenyl esters of fatty acids having 10 to 20 carbon atoms. Examples thereof include oleyl myristate, oleyl stearate, and oleyl oleate.
7. Fatty acids having 10 to 20 carbon atoms. Suitable examples include pelargonic, lauric, myrisfic, palmitic, stearic, isostearic, hydroxystearic, oleic, linoleic, ricinoleic, arachidic, behenic, and erucic acids.
8. Fatty alcohols having 10 to 20 carbon atoms. Lauryl, myristyl, cetyl, hexadecyl, stearyl, isostearyl, hydroxystearyl, oleyl, ricinoleyl, behenyl, erucyl, and 2_octyl dodecanyl alcohols are examples of satisfactory fatty alcohols.
9. Fatty alcohol ethers. Ethoxylated fatty alcohols of 10 to 20 carbon atoms including the lauryl, cetyl, stearyl, isostearyl, oleyl, and cholesterol alcohols, having attached thereto from 1 to 50 ethylene oxide groups or 1 to 50 propylene oxide groups.
10. Ether_esters such as fatty acid esters of ethoxylated fatty alcohols.
11. Lanolin and derivatives. Lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols_esters, hydrogenolysis of lanolin, ethoxylated hydrogenated lanolin, ethoxylated sorbitol lanolin, and liquid and semisolid lanolin absorption bases are illustrative of emollients derived from lanolin.
12. Polyhydric alcohol esters. Ethylene glycol mono and di_fatty acid esters, diethylene glycol mono_and di_fatty acid esters, polyethylene glycol (200_{—}6000) mono_and di_fatty acid esters, propylene glycol mono_and di_fatty acid esters, polypropylene glycol 2000 monooleate, polypropylene glycol 2000 monostearate, ethoxylated propylene glycol monostearate, glyceryl mono_and di_fatty acid esters, polyglycerol polyfatty esters, ethoxylated glyceryl monostearate, 1,3_butylene glycol monostearate, 1,3_butylene glycol distearate, polyoxyethylene polyol fatty acid ester, sorbitan fatty acid esters, and polyoxyethylene sorbitan fatty acid esters are satisfactory polyhydric alcohol esters.
13. Wax esters such as beeswax, spermaceti, myristyl myristate, stearyl stearate.
14. Beeswax derivatives, e.g. polyoxyethylene sorbitol beeswax. These are reaction products of beeswax with ethoxylated sorbitol of varying ethylene oxide content, forming a mixture of ether esters.
15. Vegetable waxes including carnauba and candelilla waxes.
16. Phospholipids such as lecithin and derivatives.
17. Sterols. Cholesterol, cholesterol fatty acid esters are examples thereof.
18. Amides such as fatty acid amides, ethoxylated fatty acid amides, solid fatty acid alkanolamides.

Amounts of the above listed emollients may range anywhere from about 0.5 to about 40% by weight of the total composition. Preferably the amounts of these emollients will range from about 2 to about 25%, optimally between about 5 and 15% by weight.

Humectants of the polyhydric alcohol_type may also be included in the compositions of this invention. The humectant aids in increasing the effectiveness of the emollients reduces scaling, stimulates removal of built_up scale and improves skin feel. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3_butylene glycol, 1,2,6_hexanetriol, ethoxylated glycerol, propoxylated glycerol and mixtures thereof. For best results the humectant is preferably glycerol. The amount of humectant may range anywhere from 0.5 to 20%, preferably between 1 and 15% by weight of the composition.

For improved lubricity, there may also be included one or more silicone oils or fluids which may be selected from a dimethyl polysiloxane, a methylphenyl polysiloxane and an alcohol_soluble silicone glycol copolymer. Preferred siloxanes include dimethyl polysiloxane (CTFA name: dimethicone), a polysiloxane end_blocked with trimethyl units and polydimethylcyclosiloxane, (CTFA name: cyclomethicone). The preferred siloxanes exhibit a viscosity from about 2 to 50 centistokes at 25 degree. C. Amounts of the silicones can range up to 30% by weight of the compositions, preferably from about 1 to about 10% by weight.

Surfactants can also be included in the compositions of this invention. These may be selected from nonionic, anionic, cationic or amphoteric emulsifying agents. They may range in amount anywhere from about 0.1 to 20% by weight. A particularly preferred anionic emulsifying agent is a dimethicone copolyol phosphate available under the trademark Pecosil.RTM. A particularly preferred nonionic emulsifying agent, especially in the formation of water_in_silicone emulsions, is cetyl dimethicone copolyol available under the trademark Abil EM_{—}90.RTM. sold by the Goldschmidt Chemical Corporation.

The emulsions of the invention can also include thickeners/viscosifiers in amounts up to about 5% by weight of the composition. As known to those skilled in the art, the precise amount of thickeners can vary depending upon the consistency and thickness of the composition which is desired. Exemplary thickeners are xanthan gum, sodium carboxymethyl cellulose, hydroxyalkyl and alkyl celluloses, and cross_linked acrylic acid polymers such as those sold by B.F. Goodrich under the Carbopol.RTM. trademark.

Waterproofing agents may also be included in the compositions of this invention. These agents may range in amount anywhere from about 0.5 to about 10% by weight. Common waterproofing agents are polymers and copolymers based on PVP and acryclic or methacrylic esters. Specific examples are PVP/Hexadecene Copolymer (Ganex V_{—}216.RTM.), PVP/Eicosene Copolymer (Ganex V_{—}220.RTM.), PVP/Ethyl Methacrylate/Methacrylic Acid Copolymer, Ammonium Acrylates Copolymer, and Polyolprepolymer_{—}2 (ex Penederm/Barnet).

Preservatives can desirably be incorporated into the cosmetic compositions of this invention to protect against the growth of potentially harmful microorganisms. While it is in the aqueous phase that microorganisms tend to grow, microorganisms can also reside in the oil phase. As such, preservatives which have solubility in both water and oil are preferably employed in the present compositions. Suitable traditional preservatives for compositions of this invention are alkyl esters of para_hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are methyl paraben, imidazolidinyl urea, sodium dehydroxyacetate, propyl paraben and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition.

Amounts of water in the composition may range anywhere from about 1 to about 99%, preferably from about 20 to about 90%, optimally between about 40 and 70% by weight.

Minor adjunct ingredients may also include fragrances, antifoam agents, bacteriostats, opacifiers and colorants, each in their effective amounts to accomplish their respective functions.

Other suitable cosmetic compositions may be employed, as known to those skilled in the art.

The entire disclosure of each patent and patent application cross-referenced or referenced herein and of each non-patent publication referenced herein is hereby incorporated herein by reference thereto, as though wholly set forth herein. Each document incorporated by reference in any of the foregoing patents, patent applications or non-patent publications is also incorporated herein in its entirety by reference thereto.

While illustrative embodiments of the invention have been described above, it is, of course, understood that many and various modifications will be apparent to those of ordinary skill in the relevant art, or may become apparent as the art develops. Such modifications are contemplated as being within the spirit and scope of the invention or inventions disclosed in this specification.

1. A mixed tea composition comprising:
a) from about 30 to about 70 weight percent of a white tea;
b) from about 15 to about 50 weight percent of a green tea;
c) from about 5 to about 40 weight percent of a yellow tea; the percentage weights being percentages of the total weight of the mixed teas in the composition.

2. A mixed tea composition according to claim 1 wherein each tea comprises an aqueous extract of the respective tea.

3. A mixed tea composition according to claim 2 wherein each tea extract has a predetermined potency as indicated by the concentration of a marker compound.

4. A mixed tea composition according to claim 3 wherein the marker compound is caffeine, optionally at a concentration of about 1 percent by weight of the extract.

5. A mixed tea composition according to claim 1 wherein the proportion of each tea is at least about 20 weight percent based upon the total weight of the mixed tea composition.

6. A mixed tea composition according to claim 3 comprising:
a) from about 40 to about 60 weight percent of the white tea;
b) from about 25 to about 35 weight percent of the green tea;
c) from about 15 to about 25 weight percent of a yellow tea; the percentages being percentages of the total weight of the mixed tea extracts in the composition.

7. A mixed tea composition comprising proportions of white tea, green tea and yellow tea effective to provide inhibition of ultaviolet-induced cell renewal approximately equivalent to that provided by an SPF sunscreen or effective to provide any of the antioxidant or antiactinic properties described quantitatively herein.

8. A human-consumable product comprising from about 0.01 to about 10 weight percent of a mixed tea composition according to claim 1.

9. A product according to claim 4 selected from the group consisting of topical cosmetic compositions, topical dermatological compositions, sunscreens, beverages and foodstuffs.
